(19) United States
(12) Patent Application Publication
(10) Pub. No.: US 2012/0301406 A1
(43) Pub. Date: Nov. 29, 2012

(54) SKIN CARE COMPOSITION WITH INDICATING FUNCTION AND METHOD OF USING THE SAME

(75) Inventors: Pei-Fen Yang, New Taipei City (TW); Magdiel Inggrid Setyawati, New Taipei City (TW)

(73) Assignee: TAIWAN TEXTILE RESEARCH INSTITUTE, New Taipei City (TW)

(21) Appl. No.: 13/232,980

(22) Filed: Sep. 14, 2011

(30) Foreign Application Priority Data
May 25, 2011 (TW) 100118282

(51) Int. Cl. A61K 8/02; A61Q 19/00; A61K 8/33

(52) U.S. Cl. 424/10.3

(57) ABSTRACT

A skin care composition with indicating function including a skin care component, a pH indicator and a buffer solution is provided. The skin care composition has a pH value less than or equal to 8. A method of using the skin care composition includes the following. The skin care composition is applied on a skin surface. After the skin care composition is applied on the skin surface for a period of time, the color of the skin care composition changes. When the color of the skin care composition changes, the skin care composition is removed from the skin surface.

### CROSS-REFERENCE TO RELATED APPLICATION

This application claims the priority benefit of Taiwan application serial no. 100118282, filed May 25, 2011. The entirety of the above-mentioned patent application is hereby incorporated by reference herein and made a part of this specification.

### BACKGROUND OF THE INVENTION

1. Field of the Invention

The invention relates to a skin care composition with indicating function and a method of using the same. The composition may be a paste, lotion, gel, cream, woven, nonwoven, or dressing that is applied to the skin. The colour change is visible through the indicator and shows the consumer that the skin care composition needs to be replaced.

2. Description of Related Art

Conventional facial masks in the market right now can be generally categorized into sheet masks, rinse off masks, mud masks and so on. Regardless of the type of the masks, most of cosmetic facial masks have their suggested time of usage. However, consumers usually tend to believe they can have better results by applying facial masks for a longer period of time which ultimately leads to excessive use of masks.

Generally, when facial masks are applied, moisture contents in facial masks evaporate over time. When a user applies a facial mask excessively, that is, when the user has the facial mask on for too long, the facial mask will absorb moisture from the skin and cause moisture loss in the skin. Additionally, when the facial mask contains an irritant ingredient, the irritant ingredient in the mask may lead to irritation and damage of the skin when the user applies the facial mask excessively, that is, when the user has the facial mask on for too long.

### SUMMARY OF THE INVENTION

The invention is directed to a skin care composition with indicating function and a method of using the same, so as to prevent a user from using the skin care composition excessively.

The invention is directed to a skin care composition with indicating function. The skin care composition includes a skin care component, a pH indicator, and a buffer solution. The skin care composition has a pH value less than or equal to 8.

In one embodiment of the invention, the skin care component includes a mask composition, a cream composition, a serum composition, or a combination thereof.

In one embodiment of the invention, the mask composition includes a sheet mask composition, a rinse off mask composition, or a mud mask composition.

In one embodiment of the invention, the sheet mask composition is coated on a substrate. The substrate includes an aqua gel substrate, a gel substrate, a biocellulose substrate, a woven substrate, or a nonwoven substrate.

In one embodiment of the invention, the skin care component accounts for 50 wt % to 99.9 wt %.

In one embodiment of the invention, the pH indicator accounts for 0.01 wt % to 0.5 wt %.

In one embodiment of the invention, the pH indicator includes a natural pH indicator or a normal pH indicator.

In one embodiment of the invention, the natural pH indicator includes an anthocyanin pigment, a litmus pigment, an alizarin pigment, a cochineal pigment, a red cabbage pigment, or a grape pigment.

In one embodiment of the invention, the normal pH indicator includes methyl red, alizarin red S, bromocresol purple, chlorophenol red, bromophenol blue, p-nitrophenol, azolitmin, phenol red, neutral red, rosolic acid, cresol red, α-naphtholphthalein, or tumaric curcumin.

In one embodiment of the invention, the buffer solution has a pH value ranging from 6 to 8.

In one embodiment of the invention, the buffer solution has a concentration ranging from 0.05 wt % to 1.5 wt %.

In one embodiment of the invention, the buffer solution includes a phosphate buffer solution, an acetate buffer solution, a glycine buffer solution, a sodium hydrogen maleate buffer solution, a bis-tris(hydroxymethyl)aminomethane buffer solution, a triethanolamine hydrochloride buffer solution, or a bis[tris(hydroxymethyl)methylamino] propane buffer solution.

In one embodiment of the invention, the buffer solution includes a sodium cacodylate/hydrochloric acid buffer solution, a sodium hydrogen maleate/sodium hydroxide buffer solution, a maleic acid/tris(hydroxymethyl)aminomethane/sodium hydroxide buffer solution, a disodium hydrogen phosphate/monosodium phosphate buffer solution, a monopotassium phosphate/sodium hydroxide buffer solution, an imidazole/hydrochloric acid buffer solution, a 2,4,6-trimethylpyridine/hydrochloric acid buffer solution, a bis-tris(hydroxymethyl)aminomethane/hydrochloric acid buffer solution, a bis[tris(hydroxymethyl)methylamino]propane/hydrochloric acid buffer solution, a triethanolamine hydrochloride/sodium hydroxide buffer solution, a sodium 5,5-diethylbarbiturate/hydrochloric acid buffer solution, an N-ethylmorpholine/hydrochloric acid buffer solution, a sodium pyrophosphate/hydrochloric acid buffer solution, a tris(hydroxymethyl)aminomethane/hydrochloric acid buffer solution, or a bicine/sodium hydroxide buffer solution.

A method of using the skin care composition with indicating function includes the following The skin care composition is applied on a skin surface, where a color of the skin care composition changes when the skin care composition is applied on the skin surface for a period of time The skin care composition is removed from the skin surface when the color of the skin care composition changes.

In one embodiment of the invention, a length of the period of time correlates to a concentration of the buffer solution.

In one embodiment of the invention, the period of time is less or equal to 30 minutes.

In one embodiment of the invention, when the skin care composition contacts with the skin surface, hydrogen ions diffuse from the skin surface to the skin care composition gradually.

In light of the foregoing, the skin care composition of the invention includes a skin care component, a pH indicator, and a buffer solution. After the user applies the skin care composition for a period of time, the pH indicator in the skin care composition changes color to notify the user for removing the skin care composition. As a consequence, the user is prevented from using the skin care composition excessively which may be unfavorable for the skin.

In order to make the aforementioned and other features and advantages of the invention more comprehensible, several embodiments accompanied with figures are described in detail below.

### BRIEF DESCRIPTION OF THE DRAWINGS

The accompanying drawings are included to provide further understanding, and are incorporated in and constitute a part of this specification. The drawings illustrate embodiments and, together with the description, serve to explain the principles of the invention.

FIG. 1 illustrates a flowchart of a method of using a skin care composition with indicating function according to an embodiment of the invention.

### DESCRIPTION OF EMBODIMENTS

The invention is directed to a skin care composition with indicating function. The skin care composition includes a skin care component, a pH indicator, and a buffer solution. The skin care composition has a pH value less than or equal to 8.

In the invention, the skin care composition with indicating function can be a mask, a cream, a serum, or a combination thereof. In other words, when the skin care composition with indicating function is a mask, a skin care component in the skin care composition is a mask composition. When the skin care composition is a cream, a skin care component in the skin care composition is a cream composition. When the skin care composition is a serum, a skin care component in the skin care composition is a serum composition. According to the present embodiment, the skin care component (i.e. the mask composition, the cream composition, the serum composition, or a combination thereof) accounts for 50 wt % to 99.9 wt %.

When the skin care composition is a mask, the mask composition in the skin care composition can be a sheet mask composition, a rinse off mask composition, or a mud mask composition. That is, the skin care composition can be a sheet mask, a rinse off mask, or a mud mask. In details, the mask composition is coated on a substrate in the sheet mask, where the substrate is an aqua gel substrate, a gel substrate, a biocellulose substrate, a woven substrate, or a nonwoven substrate. As for the rinse off mask, the rinse off mask composition is applied directly on a skin surface and rinsed off with water afterwards. As for the mud mask, the mud mask composition is applied directly on a skin surface and rinsed off with water afterwards.

Moreover, in the present embodiment, a pH indicator in the skin care composition accounts for 0.01 wt % to 0.5 wt %. The pH indicator can be a natural pH indicator or a normal pH indicator. Herein, the applicable natural pH indicators are listed in Table 1.

TABLE 1

| | Natural pH indicator |
|---|---|
| 1 | Litmus |
| 2 | Alizarin |
| 3 | Cochineal |
| 4 | Anthocyanin |
| 5 | Red Cabbage |
| 6 | Grapes |
| 7 | Red Beet |
| 8 | Blueberries |
| 9 | Carrots |
| 10 | Cherries |
| 11 | Curry Powder |
| 12 | Delphinium Petals |
| 13 | Geranium Petals |
| 14 | Horse Chestnut Leaves |
| 15 | Hydrangea |
| 16 | Morning Glories |
| 17 | Onion |
| 18 | Pansy Petals |
| 19 | Petunia Petals |
| 20 | Poison Primrose |
| 21 | Poppy Petals |
| 22 | Purple Peonies |
| 23 | Red Radish |
| 24 | Rhubarb |
| 25 | Rose Petals |
| 26 | Strawberries |
| 27 | Tea |
| 28 | Thyme |
| 29 | Turmeric |
| 30 | Tulip Petals |
| 31 | Violet Petals |

According to the present embodiment, the 1^{st} to the 6^{th} natural pH indicators, that is, an anthocyanin pigment, a litmus pigment, an alizarin pigment, a cochineal pigment, a red cabbage pigment, and a grape pigment are more favorable among the natural pH indicators illustrated in Table 1.

Herein, the applicable normal pH indicators are listed in Table 2.

TABLE 2

| | Normal pH indicator | pH |
|---|---|---|
| 1 | Methyl Violet | 0-.16 |
| 2 | Crystal Violet | 0-1.8 |
| 3 | Ethyl Violet | 0-2.4 |
| 4 | Malachite Green | 0.2-1.8 |
| 5 | Methyl Green | 0.2-1.8 |
| 6 | Cresol Red | 0.04-1.8 |
| 7 | Thymol Blue | 1.2-2.8 |
| 8 | Pentamethoxy red | 1.2-2.3 |
| 9 | Tropeolin OO | 1.3-3.2 |
| 10 | 2,4-Dinitrophenol | 2.4-4.0 |
| 11 | Methyl yellow | 2.9-4.0 |
| 12 | Methyl orange | 3.1-4.4 |
| 13 | Bromophenol blue/Tetrabromophenol blue | 3.0-4.6 |
| 14 | Congo Red | 3.0-5.0 |
| 15 | Alizarin sodium sulfonate | 3.7-5.2 |
| 16 | α-Naphthyl red | 3.7-5.0 |
| 17 | p-Ethoxychrysoidine | 3.5-5.5 |
| 18 | Bromocresol green | 4.0-5.6 |
| 19 | Resorcin Blue | 4.4-6.0 |
| 20 | Methyl red | 4.4-6.2 |
| 21 | Alzarin Red S | 4.6-6.0 |
| 22 | Bromocresol purple | 5.2-6.8 |
| 23 | Chlorophenol red | 5.4-6.8 |
| 24 | Bromophenol blue | 6.2-7.6 |
| 25 | p-Nitrophenol | 5.0-7.0 |
| 26 | Azolitmin | 5.0-8.0 |
| 27 | Phenol red | 6.4-8.0 |
| 28 | Neutral red | 6.8-8.0 |
| 29 | Rosolic acid | 6.8-8.0 |
| 30 | Cresol red | 7.2-8.8 |
| 31 | α-Naphtholphthalein | 7.3-8.7 |
| 32 | Tumaric Curcumin | 7.4-8.6 |
| 33 | Tropeolin OOO | 7.6-8.9 |
| 34 | Thymol blue | 8.0-9.6 |
| 35 | Phenolphthalein | 8.0-10.0 |
| 36 | α-Naphtholbenzein | 9.0-11.0 |
| 37 | Thymolphthalein | 9.4-10.6 |
| 38 | Nile blue | 10.1-11.1 |
| 39 | Alizarin yellow | 10.0-12.0 |
| 40 | Salicyl yellow | 10.0-12.0 |
| 41 | Diazo violet | 10.1-12.0 |
| 42 | Tropeolin | 11.0-13.0 |
| 43 | Nitramine | 11.0-13.0 |
| 44 | Poirrier's blue | 11.0-13.0 |
| 45 | Indigo carmine | 11.4-13.0 |
| 46 | Trinitrobenzoic acid | 12.0-13.4 |
| 47 | Clayton Yellow | 12.2-13.2 |

According to the present embodiment, the 20^{th} to the 32^{nd} normal pH indicators, that is, methyl red, alizarin red S, bromocresol purple, chlorophenol red, bromophenol blue, p-nitrophenol, azolitmin, phenol red, neutral red, rosolic acid, cresol red, α-naphtholphthalein, and tumaric curcumin are more favorable among the normal pH indicators listed in Table 2.

In addition, in the present embodiment, the buffer solution in the skin care composition has a pH value ranging from 6 to 8. The buffer solution has a concentration ranging from 0.05 wt % to 1.5 wt %. In one embodiment of the invention, the buffer solution includes a phosphoric acid buffer solution, an acetic acid buffer solution, a glycine buffer solution, a sodium hydrogen maleate buffer solution, a bis-tris(hydroxymethyl)aminomethane buffer solution, a triethanolamine hydrochloride buffer solution, or a bis[tris(hydroxymethyl)methylamino] propane buffer solution. For example, the applicable buffer solutions are shown in Table 3.

TABLE 3

| | Buffer solution | pH |
|---|---|---|
| 1 | Hydrochloric acid/Potassium chloride buffer | 1.1-2.2 |
| 2 | p-Toluenesulfonic acid/Sodium p-toluenesulfonate | 1.2-2.0 |
| | buffer | |
| 3 | D(+)-Tartaric acid/Sodium hydroxide buffer | 2.1-5.1 |
| 4 | Citric acid/Sodium hydroxide buffer | 2.1-6.5 |
| 5 | Glycine/Hydrochloric acid buffer | 2.2-3.6 |
| 6 | Trans-aconitic aic/Sodium hydroxide buffer | 2.5-5.7 |
| 7 | Disodium hydrogen phosphate/Citric acid buffer | 2.6-7.6 |
| 8 | Citric acid/Sodium citrate buffer | 3.0-6.2 |
| 9 | 3,3-Dimethylglutaric acid/ | 3.2-7.6 |
| | Sodium hydroxide buffer | |
| 10 | Phenylacetic acid/sodium phenylacetate buffer | 3.4-5.2 |
| 11 | Acetic acid/Sodium acetate buffer | 3.4-5.9 |
| 12 | Succinic acid/Sodium hydroxide buffer | 3.8-6.0 |
| 13 | Sodium cacodylate/ | 5.0-7.4 |
| | Hydrochloric acid buffer | |
| 14 | Sodium hydrogen maleate/Sodium hydroxide buffer | 5.2-6.8 |
| 15 | Maleic acid/tris(hydroxymethyl)aminomethane/ | 5.2-8.6 |
| | Sodium hydroxide buffer | |
| 16 | Disodium hydrogen phosphate/Monosodium phosphate | 5.8-8.0 |
| | buffer | |
| 17 | Monopotassium phosphate/Sodium hydroxide buffer | 5.8-8.0 |
| 18 | Imidazole/Hydrochloric acid buffer | 6.2-7.8 |
| 19 | 2,4,6-Trimethylpyridine/Hydrochloric acid buffer | 6.5-8.3 |
| 20 | bis-tris(hydroxymethyl)aminomethane/ | 5.8-7.2 |
| | hydrochloric acid buffer | |
| 21 | bis[tris(Hydroxymethyl)methylamino] propane/ | 6.3-9.5 |
| | Hydrochloric acid buffer | |
| 22 | Triethanolamine hydrochloride/Sodium hydroxide | 6.8-8.8 |
| | buffer | |
| 23 | Sodium 5,5-diethylbarbiturate/ | 6.8-9.2 |
| | Hydrochloric acid buffer | |
| 24 | N-ethylmorpholine/Hydrochloric acid buffer | 6.8-8.6 |
| 25 | Sodium pyrophosphate/Hydrochloric acid buffer | 6.9-8.9 |
| 26 | Tris(hydroxymethyl)aminomethane/Hydrochloric | 7.0-9.0 |
| | acid buffer | |
| 27 | Bicine/Sodium hydroxide buffer | 7.0-8.4 |
| 28 | Diethanolamine/Hydrochloric acid buffer | 7.8-9.9 |
| 29 | Boric acid/Sodium hydroxide buffer | 8.0-10.2 |
| 30 | Sodium borate/Hydrochloric acid buffer | 8.0-9.1 |
| 31 | Ammonia/Ammonium chloride buffer | 8.2-10.8 |
| 32 | Glycine/Sodium hydroxide buffer | 8.6-10.6 |
| 33 | Sodium carbonate/Sodium bicarbonate buffer | 9.1-10.8 |
| 34 | Sodium borate/Sodium hydroxide buffer | 9.2-10.8 |
| 35 | Sodium Bicarbonate/Sodium hydroxide buffer | 9.6-11 |
| 36 | Disodium hydrogen phosphate/Sodium hydroxide buffer | 10.9-12 |

Among the buffer solutions listed in Table 3, a sodium cacodylate/hydrochloric acid buffer solution, a sodium hydrogen maleate/sodium hydroxide buffer solution, a maleic acid/tris(hydroxymethyl)aminomethane/sodium hydroxide buffer solution, a disodium hydrogen phosphate/monosodium phosphate buffer solution, a monopotassium phosphate/sodium hydroxide buffer solution, an imidazole/hydrochloric acid buffer solution, a 2,4,6-trimethylpyridine/hydrochloric acid buffer solution, a bis-tris(hydroxymethyl)aminomethane/hydrochloric acid buffer solution, a bis[tris(hydroxymethyl)methylamino]propane/hydrochloric acid buffer solution, a triethanolamine hydrochloride/sodium hydroxide buffer solution, a sodium 5,5-diethylbarbiturate/hydrochloric acid buffer solution, an N-ethylmorpholine/hydrochloric acid buffer solution, a sodium pyrophosphate/hydrochloric acid buffer solution, a tris(hydroxymethyl)aminomethane/hydrochloric acid buffer solution, and a bicine/sodium hydroxide buffer solution are more favorable.

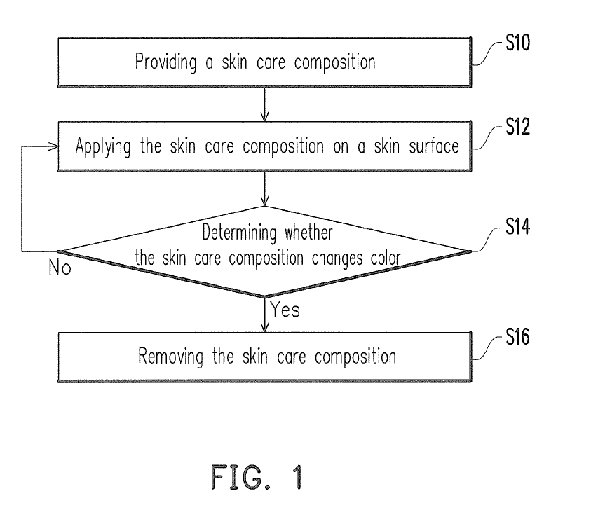

FIG. 1 illustrates a flowchart of a method of using a skin care composition with indicating function according to an embodiment of the invention. Referring to FIG. 1, firstly, step S10 is performed to provide a skin care composition with indicating function. Here, the skin care composition includes a skin care component, a pH indicator, and a buffer solution as aforementioned. The skin care composition has a pH value less than or equal to 8.

Thereafter, step S 12 is performed to apply the skin care composition on a skin surface. When the skin care composition is a sheet mask, the sheet mask is applied on the skin surface directly. When the skin care composition is a rinse off mask, a mud mask, a cream, or a serum, the skin care composition is then evenly applied on the skin surface directly.

In the present embodiment, since the skin care composition includes a pH indicator, the skin care composition changes color when the skin care composition is applied on the skin surface for a period of time. Therefore, step S14 is carried out to determine whether the skin care composition has changed color.

If the skin care composition changes color in step S14, step S16 is then performed to remove the skin care composition from the skin surface.

If the skin care composition does not change color in step 14, step S12 is carried out continuously with the skin care composition applied on the skin surface.

It should be noted that in the present embodiment, a length of time from step S12 to step S16 correlates to a concentration of the buffer solution in the skin care composition. In other words, the time for the skin care composition to change color can be adjusted through adjusting the concentration of the buffer solution in the present embodiment. In general, the time (the time to perform from S12 to S16) is less or equal to 30 minutes, but the invention is not limited thereto. For instance, when the skin care component in the skin care composition includes an irritant ingredient (i.e. an acidic ingredient), the time for the skin care composition to change color can be shortened by adjusting the concentration of the buffer solution (lowering the concentration of the buffer solution), so that the skin care composition can change color in 10-15 minutes. When the skin care component in the skin care composition includes a normal moisturizer ingredient, the time for the skin care composition to change color can be extended by adjusting the concentration of the buffer solution (increasing the concentration of the buffer solution), so that the skin care composition can change color in 25-30 minutes.

Generally, the skin surface layer has a protective acid mantle mainly constituted by sweat glands, sebaceous gland secretions, and keratinocyte cells. The protective acid mantle has a pH value ranging from 4.5 to 6.0. In step S12, after the skin care composition contacts with the skin surface, hydrogen ions in the skin (protective acid mantle) diffuse gradually from the skin surface to the skin care composition. Consequently, when the hydrogen ions from the skin enter the skin care composition and change the pH of the skin care composition, the pH indicator in the skin care composition changes color which leads to the color change of the skin care composition. Since the skin care composition in the present embodiment is pH indicative, the skin care composition can change color after contacting the skin for a period of time.

Also, as the change in pH values of the skin care composition correlates to the concentration of the buffer solution and the diffusion speed of the hydrogen ions, the time for changing the pH values of the skin care composition (for changing the color of the pH indicator) can be adjusted by adjusting the concentration of the buffer solution in the present embodiment.

Several embodiments are shown below to illustrate that the time for changing the pH values of the skin care composition (for changing the color of the pH indicator) can be adjusted through adjusting the concentration of the buffer solution. In the following embodiment, a biocellulose mask is applied, where a pH indicator in the biocellulose mask is an EV12 grape skin pigment and a buffer solution thereof is a phosphate buffer solution.

TABLE 4

| | Concentration | | | Change in pH of the mask over time (min) | | | | | | | | | | |
|---|---|---|---|---|---|---|---|---|---|---|---|---|---|---|
| | of buffer | pH | Indicator | 0 | | | | | | | | | | |
| | solution (M) | value | (%) | min | 15 | 30 | 40 | 60 | 75 | 90 | 105 | 120 | 135 | 150 |
| 1 | 0.02 | 8 | 0.05 | 8.2 | 7.5 | 7.3 | 7.2 | 6.9 | 6.7 | 6.5 | 6.4 | 6.4 | 6.2 | 6.1 |
| 2 | 0.01 | 8 | 0.05 | 8 | 7.4 | 7 | 7.1 | 6.8 | 6.4 | 6.3 | 6.2 | 6.2 | 6.1 | 6.1 |
| 3 | 0.005 | 8 | 0.05 | 7.9 | 7.2 | 6.9 | 6.9 | 6.6 | 6.3 | 6.2 | 6.1 | 6.1 | 6.1 | 6.1 |
| 4 | 0.005 | 8 | 0.05 | 7.9 | 6.9 | 6.9 | 6.7 | 6.4 | 6.3 | 6.1 | — | — | — | — |
| 5 | 0.005 | 7.5 | 0.05 | 7.4 | 6.9 | 6.7 | 6.5 | 6.3 | 6.1 | 6 | — | — | — | — |
| 6 | 0.005 | 7 | 0.05 | 6.9 | 6.5 | 6.4 | 6.2 | 6 | 6.1 | 6 | — | — | — | — |
| 7 | 0.005 | 7.5 | 0.1 | 7.3 | 6.8 | 6.6 | 6.3 | 6.2 | 6 | 6 | — | — | — | — |
| 8 | 0.005 | 7.5 | 0.05 | 7.3 | 6.8 | 6.6 | 6.4 | 6.2 | 6 | 6 | — | — | — | — |
| 9 | 0.005 | 7.5 | 0.025 | 7.3 | 6.8 | 6.6 | 6.4 | 6.3 | 6 | 6 | — | — | — | — |

As depicted in Table 4, the change in the pH values of the biocellulose mask becomes slower as the concentration of the phosphoric acid buffer solution becomes higher (i.e. 0.02 M). In other words, when the concentration of the phosphate buffer solution becomes higher, the time for the biocellulose mask to change color becomes longer. Conversely, the change in the pH values of the biocellulose mask becomes faster as the concentration of the phosphate buffer solution becomes lower (i.e. 0.005 M). In other words, when the concentration of the phosphoric acid buffer solution becomes lower, the time for the biocellulose mask to change color becomes shorter. Further, the concentration of the pH indicator has no obvious effect on the change in the pH values of the biocellulose mask.

In the embodiment shown in FIG. 5, a nonwoven mask is applied, where a pH indicator in the nonwoven mask is an EV12 grape skin pigment and a buffer solution thereof is a phosphoric acid buffer solution. The nonwoven mask is dried within approximately 15 minutes. Therefore, Table 5 shows the change in the pH values of the skin over time and the change in the pH values of the mask over time when the nonwoven mask is applied on the skin surface for 0-15 minutes.

TABLE 5

| | | | Change in pH of the skin | | | | Change in pH of the mask | | | |
|---|---|---|---|---|---|---|---|---|---|---|
| | Concentration | | over time (min) | | | | over time (min) | | | |
| Indicator | of buffer | pH | 0 | 5 | 10 | 15 | 0 | 5 | 10 | 15 |
| (%) | solution (M) | value | min | min | min | min | min | min | min | min |
| 0.1 | 0.02 | 7.5 | 5.11 | 5.21 | 5.47 | 5.55 | 7.5 | 6.4 | 6.2 | 5.8 |
| | 0.01 | | 5.11 | 5.17 | 5.41 | 5.53 | 7.5 | 6.1 | 5.9 | 5.6 |
| | 0.005 | | 5.11 | 5.14 | 5.51 | 5.51 | 7.4 | 6.0 | 5.8 | 5.6 |
| 0.1 | 0.005 | 7 | 5.15 | 5.21 | 5.35 | 5.36 | 6.9 | 5.7 | 5.5 | 5.3 |
| | | 7.5 | 5.15 | 5.29 | 5.42 | 5.42 | 7.4 | 6.0 | 5.8 | 5.5 |
| | | 8 | 5.15 | 5.35 | 5.57 | 5.57 | 7.9 | 6.9 | 6.0 | 5.6 |
| 0.2 | 0.005 | 7.5 | 5.03 | 5.33 | 5.46 | 5.46 | 7.2 | 6.0 | 5.7 | 5.5 |
| 0.1 | | | 5.03 | 5.34 | 5.46 | 5.46 | 7.4 | 6.0 | 5.8 | 5.5 |
| 0.05 | | | 5.03 | 5.32 | 5.47 | 5.46 | 7.5 | 6.0 | 6.2 | 5.5 |

As depicted in Table 5, the change in the pH values of the nonwoven mask becomes slower as the concentration of the phosphate buffer solution becomes higher. In other words, when the concentration of the phosphoric acid buffer solution becomes higher, the time for the nonwoven mask to change color becomes longer. Conversely, the change in the pH values of the nonwoven mask becomes faster as the concentration of the phosphate buffer solution becomes lower. That is, when the concentration of the phosphate buffer solution becomes lower, the time for the nonwoven mask to change color becomes shorter.

In summary, the skin care composition of the invention includes a skin care component, a pH indicator, and a buffer solution. After the user applies the skin care composition for a period of time, the pH indicator in the skin care composition changes color to notify the user for removing the skin care composition. As a consequence, the user is prevented from using the skin care composition excessively which may be unfavorable for the skin.

It will be apparent to those skilled in the art that various modifications and variations can be made to the structure of the disclosed embodiments without departing from the scope or spirit of the invention. In view of the foregoing, it is intended that the invention cover modifications and variations of this invention provided they fall within the scope of the following claims and their equivalents.

What is claimed is:

1. A skin care composition with indicating function, the skin care composition comprising:
a skin care component;
a pH indicator; and
a buffer solution,
wherein the skin care composition has a pH value less than or equal to 8.

2. The skin care composition with indicating function as claimed in claim 1, wherein the skin care component comprises a mask composition, a cream composition, a serum composition, or a combination thereof.

3. The skin care composition with indicating function as claimed in claim 2, wherein the mask composition comprises a sheet mask composition, a rinse off mask composition, or a mud mask composition.

4. The skin care composition with indicating function as claimed in claim 3, wherein the sheet mask composition is coated on a substrate and the substrate comprises an aqua gel substrate, a gel substrate, a biocellulose substrate, a woven substrate, or a nonwoven substrate.

5. The skin care composition with indicating function as claimed in claim 1, wherein the skin care component accounts for 50 wt % to 99.9 wt %.

6. The skin care composition with indicating function as claimed in claim 1, wherein the pH indicator accounts for 0.01 wt % to 0.5 wt %.

7. The skin care composition with indicating function as claimed in claim 1, wherein the pH indicator comprises a natural pH indicator or a normal pH indicator.

8. The skin care composition with indicating function as claimed in claim 7, wherein the natural pH indicator comprises an anthocyanin pigment, a litmus pigment, an alizarin pigment, a cochineal pigment, a red cabbage pigment, or a grape pigment.

9. The skin care composition with indicating function as claimed in claim 7, wherein the normal pH indicator comprises methyl red, alizarin red S, bromocresol purple, chlorophenol red, bromophenol blue, p-nitrophenol, azolitmin, phenol red, neutral red, rosolic acid, cresol red, α-naphtholphthalein, or tumaric curcumin.

10. The skin care composition with indicating function as claimed in claim 1, wherein the buffer solution has a pH value ranging from 6 to 8.

11. The skin care composition with indicating function as claimed in claim 1, wherein the buffer solution has a concentration ranging from 0.05 wt % to 1.5 wt %.

12. The skin care composition with indicating function as claimed in claim 1, wherein the buffer solution comprises a phosphate buffer solution, an acetate buffer solution, a glycine buffer solution, a sodium hydrogen maleate buffer solution, a bis-tris(hydroxymethyl)aminomethane buffer solution, a triethanolamine hydrochloride buffer solution, or a bis[tris(hydroxymethyl)methylamino] propane buffer solution.

13. The skin care composition with indicating function as claimed in claim 12, wherein the buffer solution comprises a sodium cacodylate/hydrochloric acid buffer solution, a sodium hydrogen maleate/sodium hydroxide buffer solution, a maleic acid/tris(hydroxymethyl)aminomethane/sodium hydroxide buffer solution, a disodium hydrogen phosphate/monosodium phosphate buffer solution, a monopotassium phosphate/sodium hydroxide buffer solution, an imidazole/chloride hydrogen buffer solution, a 2,4,6-trimethylpyridine/hydrochloric acid buffer solution, a bis-tris(hydroxymethyl)aminomethane/hydrochloric acid buffer solution, a bis[tris(hydroxymethyl)methylamino]propane/hydrochloric acid buffer solution, a triethanolamine hydrochloride/sodium hydroxide buffer solution, a sodium 5,5-diethylbarbiturate/chloride hydrogen buffer solution, an N-ethylmorpholine/hydrochloric acid buffer solution, a sodium pyrophosphate/hydrochloric acid buffer solution, a tris(hydroxymethyl)aminomethane/hydrochloric acid buffer solution, or a bicine/sodium hydroxide buffer solution.

14. A method of using a skin care composition with indicating function, the method comprising:
providing a skin care composition with indicating function as claimed in claim 1;
applying the skin care composition on a skin surface, wherein a color of the skin care composition changes when the skin care composition is applied on the skin surface for a period of time; and
removing the skin care composition from the skin surface when the color of the skin care composition changes.

15. The method of using the skin care composition with indicating function as claimed in claim 14, wherein a length of the period of time correlates to a concentration of the buffer solution.

16. The method of using the skin care composition with indicating function as claimed in claim 14, wherein the period of time is less or equal to 30 minutes.

17. The method of using the skin care composition with indicating function as claimed in claim 14, wherein when the skin care composition contacts with the skin surface, hydrogen ions diffuse from the skin surface to the skin care composition gradually.
